(19) United States
(12) Patent Application Publication
(10) Pub. No.: US 2014/0316291 A1
(43) Pub. Date: Oct. 23, 2014

(54) MEASUREMENT DEVICE, EVALUATING METHOD, AND EVALUATION PROGRAM

(71) Applicant: OMRON HEALTHCARE CO., LTD., Muko-shi, Kyoto (JP)

(72) Inventors: Toshiyuki Osaki, Kyoto (JP); Naoki MoriI, Kyoto (JP); Toshihiko Ogura, Kyoto (JP); Takashi Honda, Aichi (JP)

(73) Assignee: OMRON HEALTHCARE CO., LTD., Muko-shi, Kyoto (JP)

(21) Appl. No.: 14/354,216

(22) PCT Filed: Oct. 9, 2012
(86) PCT No.: PCT/JP2012/076110

§ 371 Date: Apr. 25, 2014

(30) Foreign Application Priority Data
Oct. 28, 2011 (JP) 2011-237573

(51) Int. Cl. A61B 5/00; A61B 5/022; A61B 5/021

(52) U.S. Cl. CPC A61B 5/7278; A61B 5/02116; A61B 5/022; A61B 5/7282; A61B 5/6824; A61B 5/6828; A61B 5/742
USPC 600/492

(57) ABSTRACT

A measurement device includes a blood pressure measurement unit that measures blood pressures in the upper and lower limbs, a pulse wave measurement unit that measures pulse waves in the upper and lower limbs, a first index calculation unit that calculates an ABI by calculating the ratio of the blood pressures in the upper and lower limbs, a second index calculation unit that calculates a second index used for evaluation of the ABI using the pulse waves in the upper and lower limbs, an evaluation unit that evaluates the reliability of the ABI using the ABI and the second index, and an output unit that outputs the ABI along with the result of evaluation.

### BACKGROUND OF THE INVENTION

1. Field of the Invention

The present invention relates to measurement devices, evaluating methods, and evaluation programs, and more particularly to measurement devices for calculating the ABI (ankle brachial blood pressure index), and methods and programs for evaluating the same.

2. Description of the Related Art

The ABI (ankle brachial blood pressure index) is the ratio of blood pressures in the lower and upper limbs, which indicates the presence or absence of arteriostenosis or the degree of arteriostenosis.

For example, as disclosed in JP 2004-261319A, the ABI has been conventionally obtained by measuring the blood pressures in the lower and upper limbs of a subject in the supine position with a blood pressure measurement device and then calculating the ratio of these pressures.

However, if the subject suffers from severe arterial calcification, such a conventional measuring method may not be able to accurately measure blood pressures due to insufficient compression. This tends to compromise the reliability of the ABI calculated from the blood pressure measurements.

Furthermore, if the subject is afflicted with unstable pulse amplitude due to arrhythmia or small pulse amplitude due to angiostenosis, the conventional method may not accurately measure blood pressures. This also compromises the reliability of the ABI calculated from the blood pressure measurements.

### SUMMARY OF THE INVENTION

Accordingly, preferred embodiments of the present invention provide a measurement device, an evaluating method, and an evaluation program for evaluating the reliability of the ABI calculated from blood pressure measurements.

According to a preferred embodiment of the present invention, a measurement device for measuring biological values and calculating an ABI (Ankle Brachial Blood Pressure Index) as an index of arteriostenosis from the biological values includes a first cuff configured to be worn on an upper limb of a subject; a second cuff configured to be worn on a lower limb of the subject; a first sensor that detects an internal pressure of the first cuff; a second sensor that detects an internal pressure of the second cuff; an adjustment unit that adjusts the internal pressures of the first and second cuffs; an arithmetic unit connected to the first and second sensors and configured to measure the biological values from detection values detected by the sensors and performing operations to calculate an index using the biological values; and an output device connected to the arithmetical unit to output results of the operations performed by the arithmetic unit. The arithmetic unit includes a blood pressure measurement device that measures a blood pressure in the upper limb using the detection values obtained by the first sensor and measures a blood pressure in the lower limb using the detection values obtained by the second sensor, a pulse wave measurement device that measures pulse waves in the upper limb using the detection values obtained by the first sensor and measures pulse waves in the lower limb using the detection values by the second sensor, a first calculation device that calculates an ABI by calculating the ratio of the blood pressures in the upper and lower limbs, a second calculation device that calculates a determination index used for evaluation of the ABI using the pulse waves in the upper and lower limbs, an evaluation device that evaluates the reliability of the ABI using the ABI calculated by the first calculation device and the determination index calculated by the second calculation device, and an output device that causes the output device to output the ABI along with the result of evaluation by the evaluation device.

Preferably, the evaluation device evaluates the reliability of the ABI by determining whether or not each of the ABI and the determination index is in a prescribed range thereof.

More preferably, the evaluation device evaluates the reliability of the ABI as high if the ABI and the determination index are both in their respective prescribed ranges and evaluates the reliability of the ABI as low if otherwise.

Preferably, the evaluation device evaluates the reliability of the ABI as high if the ABI is in a prescribed range from the determination index and evaluates the reliability of the ABI as low if otherwise.

Preferably, the determination index uses at least one of: % MAP (a normalized pulse wave area), which is an index representing the sharpness of a pulse wave; an UT (upstroke time), which is an index representing a rising feature value of an ankle pulse wave; a pulse wave amplitude; and an index value representing a lower limb-upper limb pulse wave transfer function, which is a function for transfer of a pulse wave from the upper limb to the lower limb.

More preferably, the determination index is calculated by combining at least two of % MAP, UT, pulse amplitude, and an index value representing a lower limb-upper limb pulse wave transfer function.

More preferably, the determination index is calculated by combining an index value representing a lower limb-upper limb pulse wave transfer function with at least one of % MAP, UT, and pulse amplitude.

Preferably, the output device causes the output device to output an estimate value of the ABI calculated by second calculation device along with the ABI.

According to another preferred embodiment of the present invention, an evaluation method for evaluating the reliability of an ABI (Ankle Brachial Blood Pressure Index) as an index of arteriostenosis, the ABI being calculated from biological values, includes the steps of obtaining an ABI calculated as the ratio of blood pressures in an upper limb and a lower limb of a subject; calculating a determination index used for evaluation of the ABI using pulse waves in the upper and lower limbs; evaluating the reliability of the ABI using the ABI and the determination index; and outputting the ABI to an output device along with a result of evaluation.

In still another preferred embodiment of the present invention, an evaluation program for causing a computer to perform operations to evaluate the reliability of an ABI (Ankle Brachial Blood Pressure Index) as an index of arteriostenosis, the ABI being calculated from biological values, causes the computer to perform the steps of obtaining an ABI calculated as the ratio of blood pressures in an upper limb and a lower limb of a subject; calculating a determination index used for evaluation of the ABI using pulse waves in the upper and lower limbs; evaluating the reliability of the ABI using the ABI and the determination index; and outputting the ABI to an output device along with a result of evaluation.

According to various preferred embodiments of the present invention, the reliability of the ABI calculated from measurements of blood pressures is simply and accurately evaluated.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 shows an exemplary configuration of a measurement device according to a preferred embodiment of the present invention.

FIG. 2 is a block diagram showing a specific example of the functional configuration of the measurement device in FIG. 1.

FIG. 3 is a graph showing a correlation between the ABI and % MAP.

FIG. 4 is a graph showing a correlation between the ABI and UT.

FIG. 5 is a graph showing a correlation between the ABI and pulse amplitude.

FIG. 6 is a graph showing a correlation between the ABI and the EABI, which is a second index, calculated from pulse waves.

FIG. 7 is a diagram showing detailed measurement results of the subject from whom the measurements denoted by P1 in FIG. 6 were taken.

FIG. 8 is a diagram showing detailed measurement results of the subject from whom the measurements denoted by P2 in FIG. 6 were taken.

FIG. 9 is a diagram showing detailed measurement results of the subject from whom the measurements denoted by P3 in FIG. 6 were taken.

FIGS. 10A and 10B illustrate graphs that show measurement results of pulse waves in the right ankle in FIG. 10A and the left ankle in FIG. 10B of a healthy subject.

FIG. 11 is a graph showing the step response for the right upper arm to the right ankle (the right step response) calculated from the pulse waves measured in the right ankle of FIG. 10A and the pulse waves measured in the right upper arms.

FIG. 12 is a graph showing the step response for the left upper arm to the left ankle (the left step response) calculated from the pulse waves measured in the left ankle of FIG. 10B and the pulse waves measured in the left upper arms.

FIG. 13 is a graph comparing the right step response of FIG. 11 and the left step response of FIG. 12.

FIG. 14 is an X-ray image showing the arterial condition of a patient with arteriosclerosis obliterans who is a measurement subject.

FIGS. 15A and 15B illustrate graphs showing measurement results of pulse waves in the right upper arm in FIG. 15A and the right ankle FIG. 15B of the patient shown in FIG. 14.

FIGS. 16A and 16B illustrate graphs showing measurement results of pulse waves in the left upper arm in FIG. 16A and the left ankle FIG. 16B of the patient shown in FIG. 14.

FIG. 17 illustrates a graph showing the right step response calculated from pulse waves measured in the right upper arm and the right ankle shown in FIGS. 15A and 15B.

FIG. 18 illustrates a graph showing the left step response calculated from pulse waves measured in the left upper arm and the left ankle shown in FIGS. 16A and 16B.

FIG. 19 is a graph comparing the right step response of FIG. 17 with the left step response of FIG. 18.

FIG. 20 is a schematic diagram of the Avolio Model.

FIG. 21 is a table showing the degrees of stenosis created in the segments designated by the element numbers 82, 104, and 111 (circled in FIG. 20) in the Avolio Model that were used by the inventors for performing calculations.

FIG. 22 is a graph plotting the results of the calculations performed by the inventors.

FIG. 23 is a graph describing the upper area, the ratio of the upper area to the lower area, and the maximum value defined in a step response interval.

FIG. 24 is a graph showing a correlation between the ABI and the upper area of the step response.

FIG. 25 is a graph showing a correlation between the ABI and the ratio of the upper area to the lower area of the step response.

FIG. 26 is a graph showing a correlation between the ABI and the maximum value of the step response interval.

FIG. 27 is a graph showing a correlation between the ABI and the EABI.

FIG. 28 is a flowchart representing a specific example of the operational flow that occurs in the measurement device.

FIG. 29 is a flowchart representing a specific example of the operation in Step S113 of FIG. 28.

### DETAILED DESCRIPTION OF THE PREFERRED EMBODIMENTS

Preferred embodiments of the present invention will be described hereinafter with specific reference to the attached drawings. The same numerals refer to the same components and elements throughout the description and the drawings, such that the designations and functions of these elements are also identical.

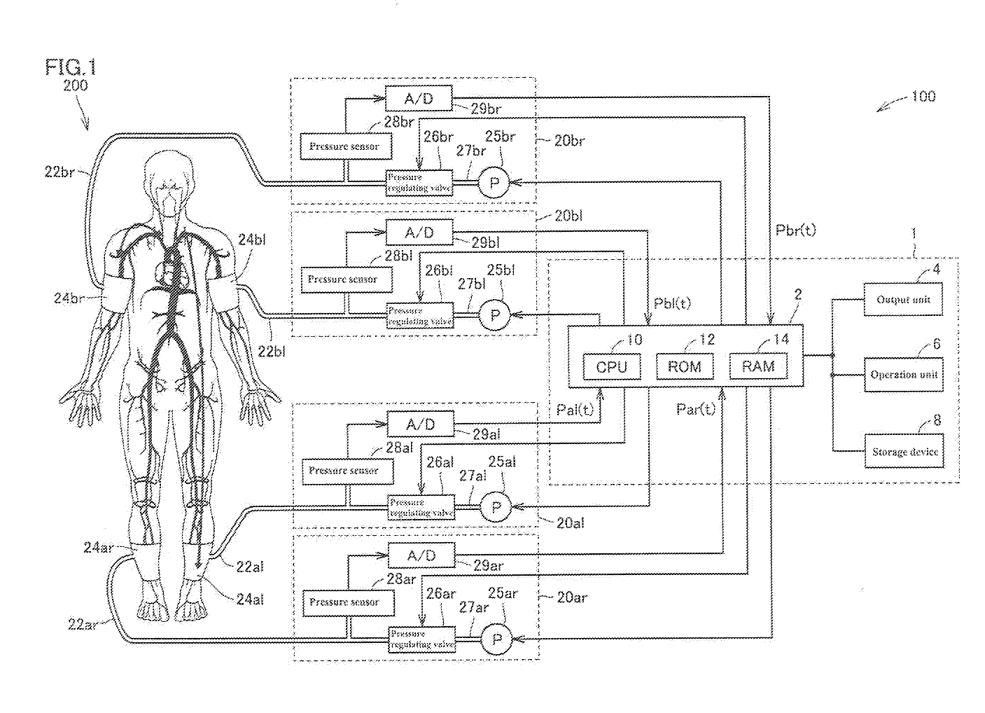

FIG. 1 shows an exemplary configuration of a measurement device 100 according to a preferred embodiment of the present invention.

Referring to FIG. 1, the measurement device 100 preferably includes an information processing unit 1, four detection units 20ar, 20al, 20br, and 20bl, and four cuffs 24ar, 24al, 24br, and 24bl.

The cuffs 24br, 24bl, 24ar, and 24al are worn on respective extremities of a subject 200. Specifically, they are respectively worn on the right upper arm (right upper limb), left upper arm (left upper limb), right ankle (right lower limb), and left ankle (left lower limb). As used herein, the term “extremity” refers to a site on any of the four limbs, and may be a wrist, a fingertip, or the like. Throughout the specification, the cuffs 24ar, 24al, 24br, and 24bl will be collectively referred to as “cuffs 24” unless there is a need to distinguish between individual cuffs.

The detection units 20ar, 20al, 20br, and 20bl each include hardware necessary to detect pulse waves in an extremity of the subject 200. As all the detection units 20ar, 20al, 20br, and 20bl may have an identical configuration, they will be collectively referred to as “detection units 20” unless there is a need to distinguish between individual units.

The information processing unit 1 includes a control unit 2, an output unit 4, an operation unit 6, and a storage device 8. The control unit 2 is a device that performs overall control of the measurement device 100 and is preferably implemented by a computer that includes a CPU (central processing unit) 10, a ROM (read only memory) 12, and a RAM (random access memory) 14.

The CPU 10 corresponds to an arithmetic processing unit, reads a program previously stored in the ROM 12, and executes the program while using the RAM 14 as the work memory.

Additionally, the output unit 4, the operation unit 6, and the storage device 8 are connected to the control unit 2. The output unit 4 outputs measured pulse waves, the result of analysis of pulse waves, and the like. The output unit 4 may be, for example, a display device implemented by LEDs (light emitting diodes) or an LCD (liquid crystal display), or a printer (driver).

The operation unit 6 is adapted to receive instructions from a user. The storage device 8 is adapted to hold various types of data and programs. The CPU 10 of the control unit 2 reads data and programs stored in the storage device 8 as well as performing writing to the storage device 8. For example, the storage device 8 may be implemented by a hard disk drive, nonvolatile memory (e.g., a flash memory), or a removable recording medium.

The specific configuration of each of the detection units 20 is described hereinafter. The detection unit 20br detects pulse waves in the right upper arm by adjusting and detecting the internal pressure of the cuff 24br worn by the subject 200 on the right upper arm (hereinafter “cuff pressure”). The cuff 24br contains a fluid bag (not shown), such as an air bag.

The detection unit 20br includes a pressure sensor 28br, a pressure regulating valve 26br, a pressure pump 25br, an A/D (analog-to-digital) converter 29br, and a tube 27br. The cuff 24br is connected to the pressure sensor 28br and the pressure regulation valve 26br via the tube 22br.

The pressure sensor 28br is a device that detects pressure fluctuation transmitted through the tube 22br and may be implemented, for example, on a semiconductor chip made of single crystal silicon or any other suitable material. A signal representing the pressure fluctuation detected by the pressure sensor 28br is converted to a digital signal by the A/D converter 29br and sent to the control unit 2 as a pulse wave signals pbr(t).

The pressure regulating valve 26br is interposed between the pressure pump 25br and the cuff 24br and maintains the pressure used to pressurize the cuff 24br in a predetermined range during measurement. The pressure pump 25br operates in accordance with a detection instruction from the control unit 2 to supply air to the fluid bag (not shown) in the cuff 24br in order to pressurize the cuff 24br.

This pressurization of the fluid bag causes the cuff 24br to press against the measurement site, such that pressure variations corresponding to pulse waves in the right upper arm are transmitted to the detection unit 20br via the tube 22br. The detection unit 20br detects the pulse waves at the right upper arm by detecting the pressure variations transmitted thereto.

Similarly, the detection unit 20bl includes a pressure sensor 28bl, a pressure regulating valve 26bl, a pressure pump 25bl, an A/D converter 29bl, and a tube 27bl. The cuff 24bl is connected to the pressure sensor 28bl and the pressure regulation valve 26bl by the tube 22bl.

Likewise, the detection unit 20ar includes a pressure sensor 28ar, a pressure regulating valve 26ar, a pressure pump 25ar, an A/D converter 29ar, and a tube 27ar. The cuff 24ar is connected to the pressure sensor 28ar and the pressure regulating valve 26ar via the tube 22ar.

Similarly, the detection unit 20al includes a pressure sensor 28al, a pressure regulating valve 26al, a pressure pump 25al, an A/D converter 29al, and a tube 27al. The cuff 24al is connected to the pressure sensor 28al and the pressure regulating valve 26al via the tube 22al.

As the functions of the components in the detection units 20bl, 20ar, and 20al are identical to those of the detection unit 20br, detailed description thereof is omitted. Likewise, reference symbols, such as “ar” and “br,” are omitted from the description of the components in the detection units 20 hereinafter unless there is a need to distinguish between them.

Note that although a configuration that detects pulse waves using the pressure sensors 28 is described in this preferred embodiment, it is possible to use a configuration that detects pulse waves using arterial volume sensors (not shown). In this case, such arterial volume sensors may include a light-emitting device that irradiates an artery and a light-receiving element that receives the light irradiated by the light-emitting device after it is transmitted through or reflected by the artery. An alternative configuration may include a plurality of electrodes that feed a minute constant current to the measurement site of the subject 200 so as to detect the voltage variations caused by the variations in impedance (bioelectrical impedance) that occur in accordance with the pulse wave propagation.

The measurement device 100 of this preferred embodiment uses the blood pressures measured in the upper and lower limbs to calculate the ABI (ankle brachial blood pressure index), which is the ratio of these pressures. The ABI is used in the preferred embodiment as a first index that indicates the presence or absence of stenosis in the arteries or the degree of stenosis in the subject.

As mentioned above, it is known that blood pressure values are susceptible to calcification of the arteries. Also, the subject may have unstable pulse amplitude due to arrhythmia or small pulse amplitude due to angiostenosis and it is also known that blood pressure values are susceptible to these conditions.

In contrast, since the wave pulses are calculated based on waveforms for several heartbeats, it is less susceptible to the aforementioned conditions. Accordingly, the measurement device 100 calculates a second index from the wave pulses measured in the upper and lower limbs and uses the second index to evaluate the reliability of the ABI, which is calculated as the first index. The second index is used as an index of arteriostenosis that can be compared with the ABI. The second index will be described in further detail below.

The measurement device 100 outputs the result of evaluation as well as the ABI, which is calculated as the first index.

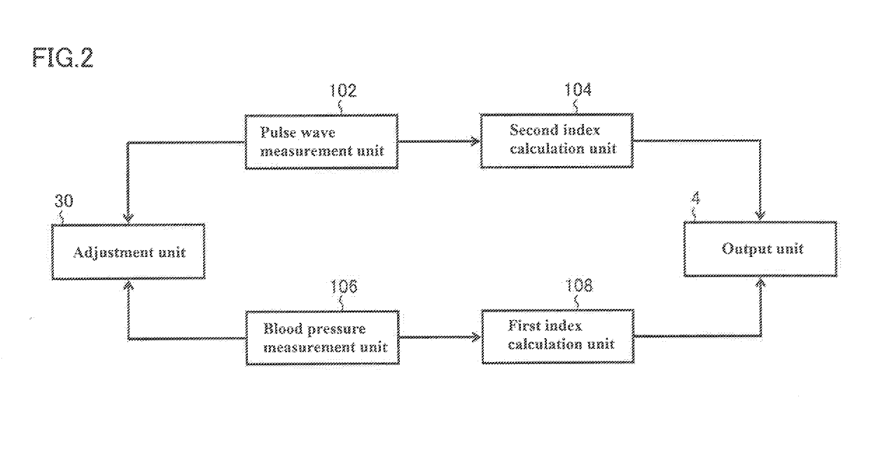

FIG. 2 is a block diagram showing a specific example of the functional configuration of the measurement device 100 to perform the foregoing operation.

The functions shown in FIG. 2 are mainly implemented on the CPU 10 as the CPU 10 reads out a program stored in the ROM 12 and executes the program while using the RAM 14 as the work memory. It should be noted, however, that at least some of the functions may be implemented by the system configuration shown in FIG. 1 or other hardware, such as electric circuitry, for example.

With reference to FIG. 2, the measurement device has various functions implemented therein, including an adjustment unit 30, a pulse wave measurement unit 102, a second index calculation unit 104 that calculates the aforementioned second index, a blood pressure measurement unit 106, a first index calculation unit 108 that calculates the aforementioned first index, an evaluation unit 110, and an output unit 4.

The adjustment unit 30 is a functional unit that adjusts the pressure inside the cuffs 24. The functionality of the adjustment unit 30 may be implemented, for example, by the pressure pump 25 and the pressure regulating valve 26 shown in FIG. 1.

The pulse wave measurement unit 102 is connected to the adjustment unit 30 and the A/D converter 29 to perform processing necessary to measure the pulse waves (PVR) in the extremity. The pulse wave measurement unit 102 adjusts the pressure inside the cuffs 24 by providing a command signal to the adjustment unit 30 and receives cuff pressure signals Par(t), Pal(t), Pbr(t), and Pbl(t) detected in response to the command signal. Subsequently, pulse waveforms for multiple heartbeats are obtained in each extremity by recording the received cuff pressure signals Par(t), Pal(t), Pbr(t), and Pbl(t) in time series. This pulse wave measurement is performed for a predetermined duration of time (for example, approximately 10 seconds).

The evaluation unit 110 evaluates the reliability of the ABI, i.e., the first index, with the second index and pass the result of the evaluation to the output unit 4.

The evaluation unit 110 may carry out any one of a variety of evaluation methods. In an exemplary method, the evaluation unit 110 stores in advance a normal range of the ABI and a normal range of the second index, compares the calculated ABI and the second index with the respective normal ranges, and evaluates the reliability of the ABI as high if both indices are in the normal ranges and evaluates it as low if they are outside of the normal ranges.

As another example, the evaluation unit 110 may compare the calculated ABI with the second index and determine that the reliability of the ABI is high if the indices coincide with each other or the ABI is in a predetermined range from the second index and determine that the reliability of the ABI is low if it is otherwise.

The following describes the foregoing second index. In addition to pulse amplitude, examples of indices of arteriostenosis using pulse waves include the sharpness of a pulse wave called % MAP (a normalized pulse wave area). % MAP is calculated, for example, as the ratio of M to H (% MAP=M/H×100), where M is the height from the minimal blood pressure when the pulse wave area is leveled and H is the peak height of the pulse wave (i.e., pulse pressure). The % MAP index value increases in the presence of arteriostenosis or arterial occlusion.

Another example is an index called a UT (upstroke time) indicating a rising feature value of an ankle pulse wave. The UT is calculated as the rising period of the ankle pulse wave from the rising point to the peak. If arteriostenosis or arterial occlusion exists in the subject, this period is extended, thus increasing the UT index value.

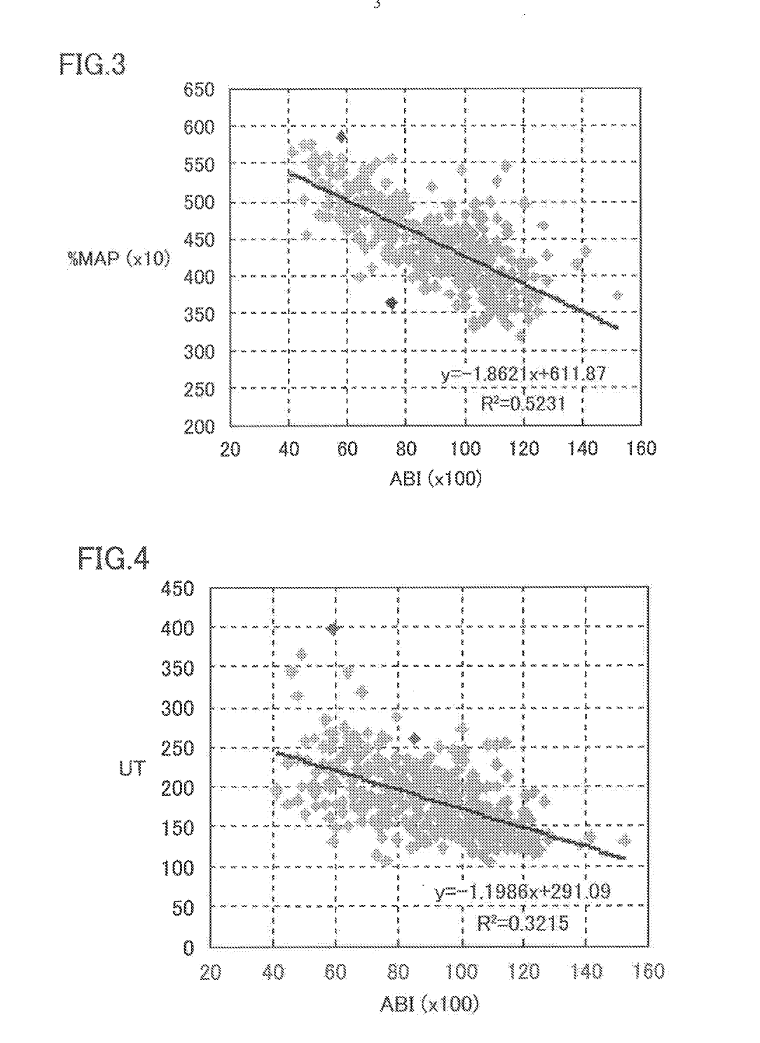

The inventors of the present application examined the correlation between these indices and the ABI or the first index. FIGS. 3-5 are graphs showing correlations between the ABI and % MAP, UT, and pulse amplitude, respectively. These values were obtained by measuring the blood pressures and pulse waves of 200 adult males and females to calculate their ABIs and % MAP, UT, and pulse amplitude.

FIGS. 3-5 verify that a certain degree of correlation exists between the ABI and any of % MAP, UT, and pulse amplitude, respectively. It is therefore thought that any of % MAP, UT, and pulse amplitude may be used as the second index to evaluate the reliability of the ABI or the first index. Alternatively, it is also thought possible to use a combination of at least two of % MAP, UT, and pulse amplitude as the second index in order to enhance the correlation.

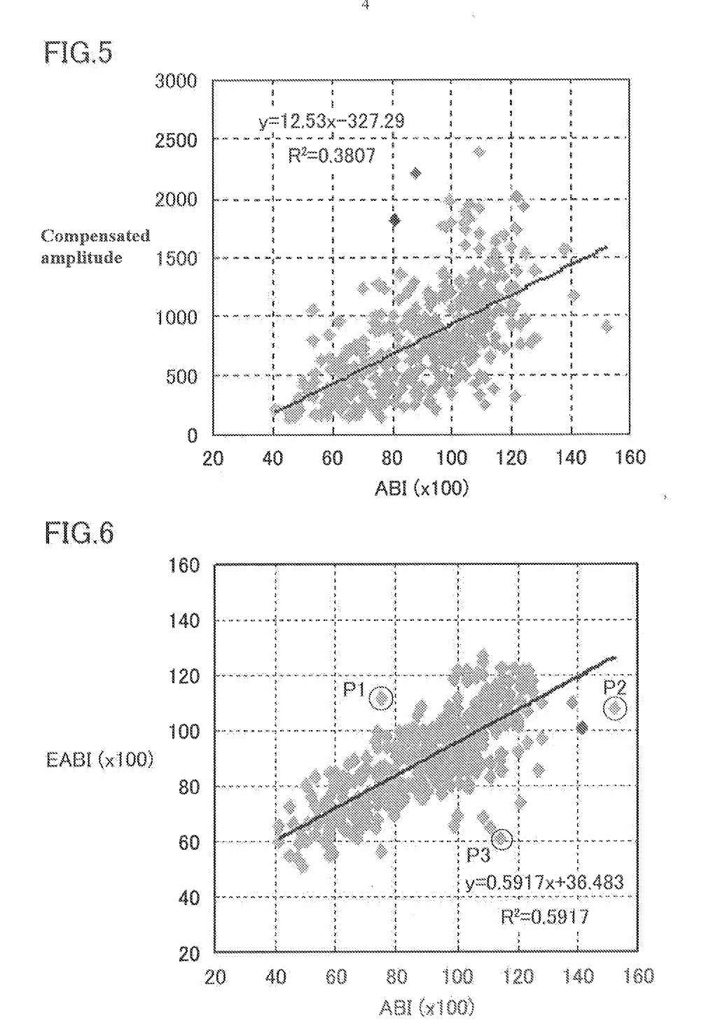

As an example, the inventors of the present application calculated a value by multiplying each of % MAP (A), UT (B), and pulse amplitude (C) by a conversion factor, as a second index (the EABI), and examined the correlation between this index and the ABI, i.e., the first index. In particular, the second index was obtained according to the formula, EABI=aA+bB+cC+d (where a-d are coefficients), so as to compare this index with the ABI. FIG. 6 is a graph showing a correlation between the ABI and the EABI.

FIG. 6 verifies that a certain degree of correlation exists between the ABI and the second index calculated by combining % MAP (A), UT (B), and pulse amplitude (C), and FIG. 6 also verifies that this correlation is stronger than the correlation between the ABI and any one of % MAP, UT, and pulse amplitude.

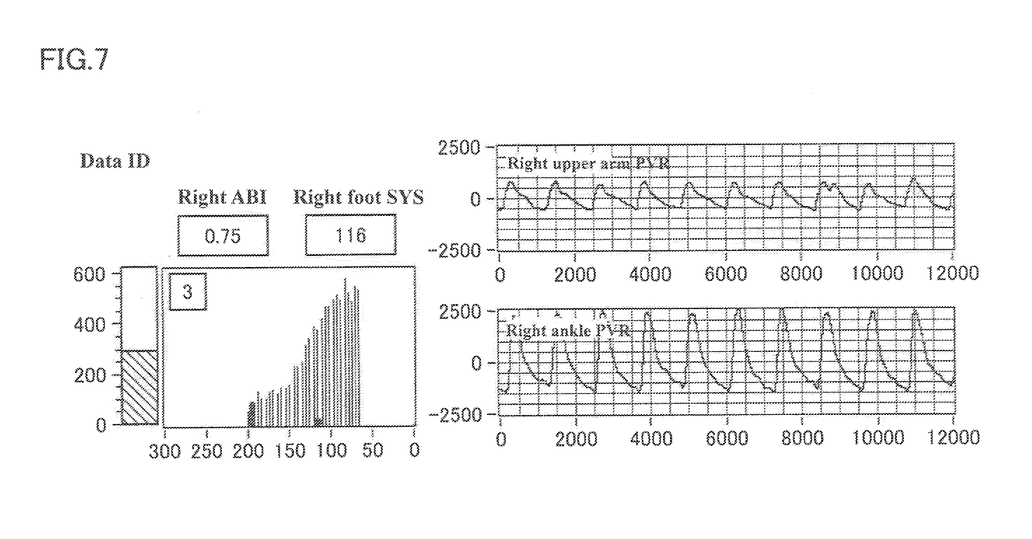

As indicated by P1-P3 in FIG. 6, however, there are several measurements that greatly deviate from the regression line. FIGS. 7-9 are diagrams showing detailed measurement results of the subjects from whom the measurements denoted by P1-P3 were taken. Each of FIGS. 7-9 shows the ABI calculated from the blood pressure values of the right upper arm and the right ankle (the right ABI), the maximal blood pressure value obtained from the right ankle pressure value, and sphygmograms taken from the right upper arm and the right ankle of the respective subject. Additionally, FIGS. 7-9 each include a time-variation graph showing the pulse wave amplitude measured over time.

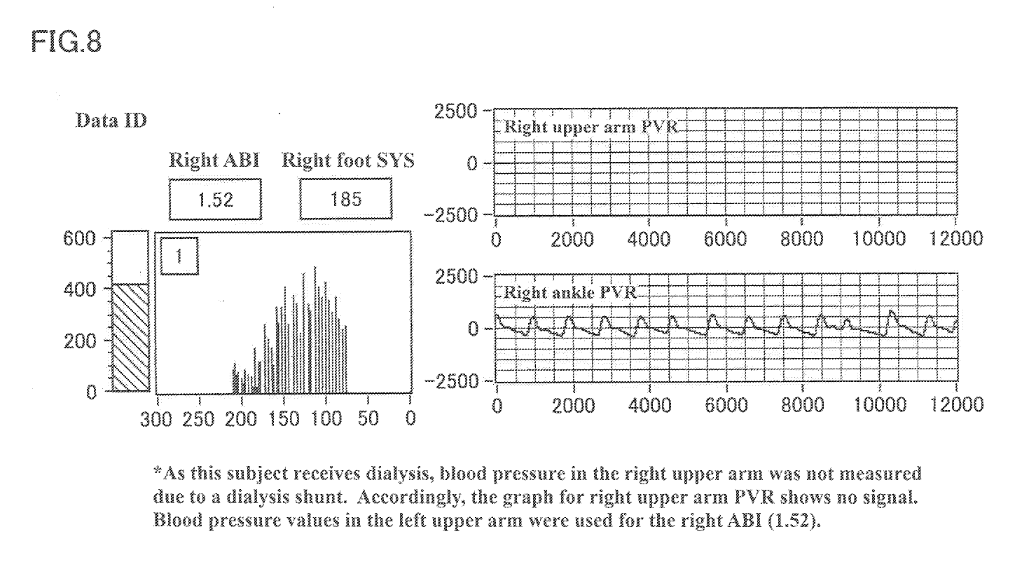

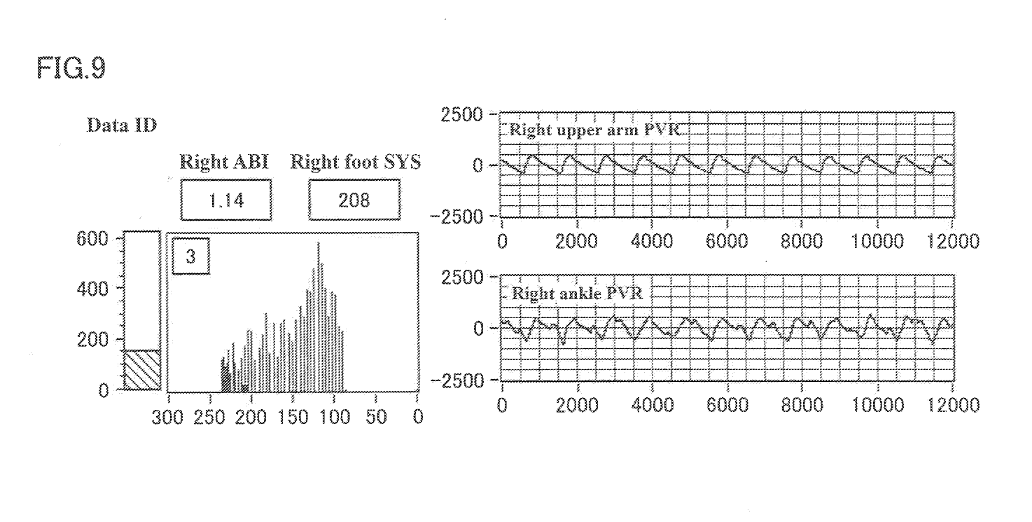

In the example of FIG. 7, as the time-variation graph of the pulse wave amplitude is incomplete, it is possible that the blood pressure in the right ankle was not accurately measured. Also, in the examples of FIGS. 8 and 9, the time-variation graphs of the pulse wave amplitude show erratic or uneven measurement patterns, indicating the possibility of inaccurate pressure measurements in the right ankle.

Based on the foregoing observation, those measurement values that greatly deviate from the regression line may be attributable to inaccurate pressure measurement. For this reason, the correlation is likely to be even stronger if these cases are excluded. In other words, it is verified that one or more of % MAP, UT, and pulse amplitude can safely be used as the second index.

Another possible index that may be used as the second index is a function for transfer of a pulse wave from the upper limb to the lower limb (a lower limb-upper limb pulse wave transfer function). This may serve as the second index because, in a transfer function where an upper limb wave pulse is the input to the system (the vascular paths) and a lower limb wave pulse is the output from the system, the presence of angiostenosis in the system is thought to affect the step response. More specifically, it is thought that this step response may be used to evaluate the reliability of the ABI, which is calculated as the first index.

To verify this, the inventors of the present application measured the pulse waves of a healthy subject and a patient with arteriosclerosis obliterans (ASO) and calculated their step responses.

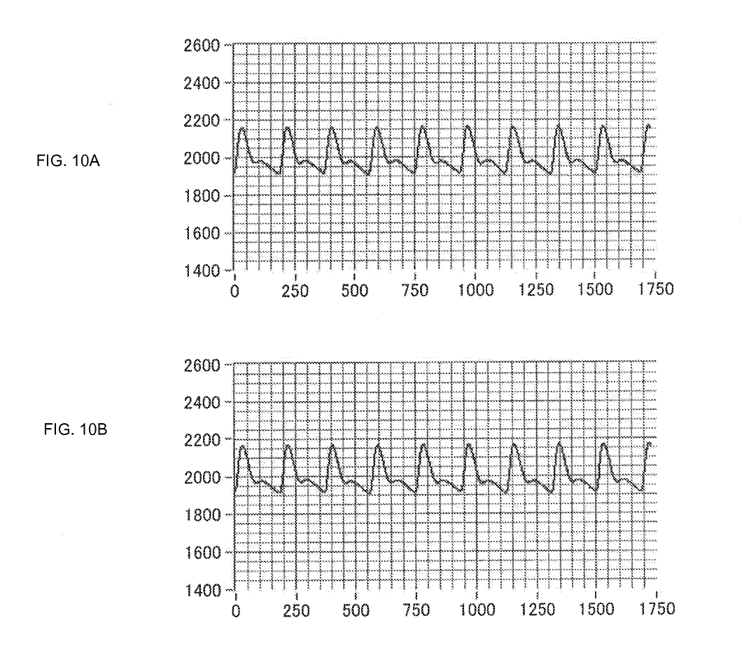

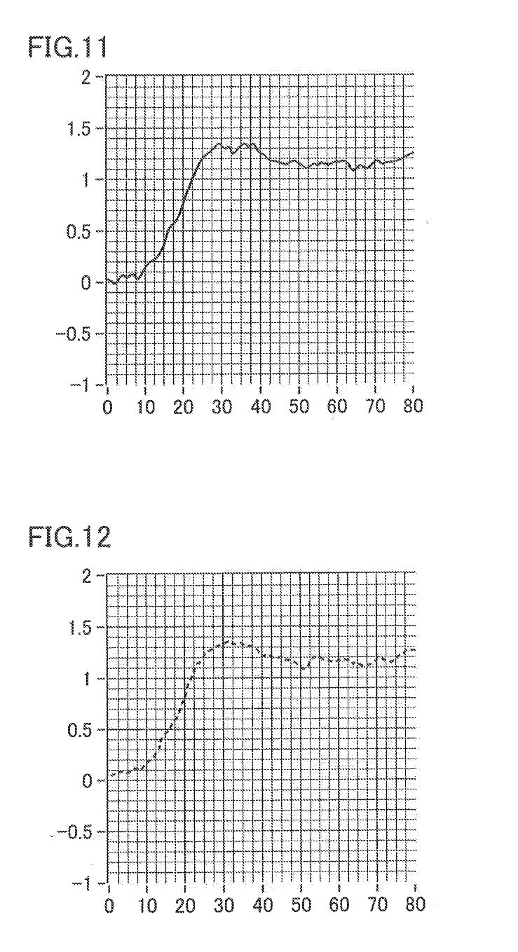

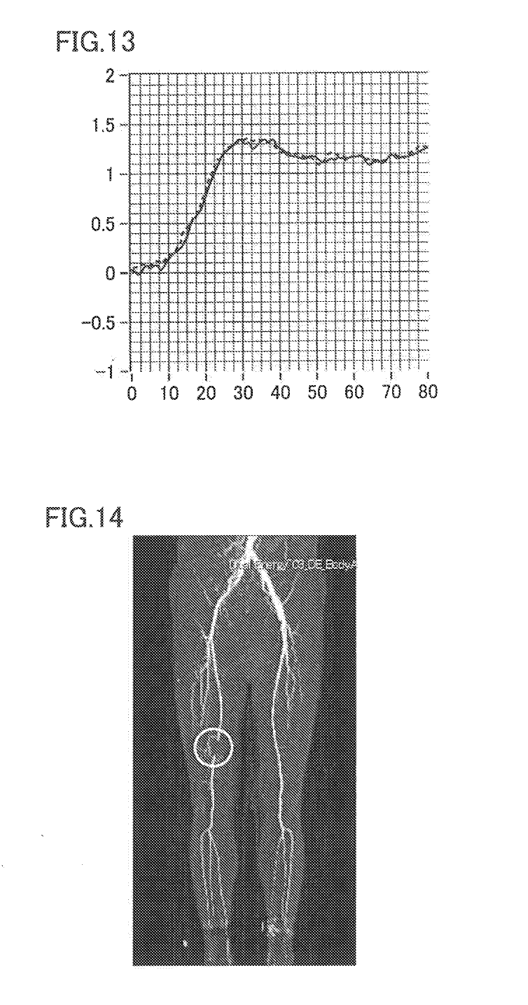

FIGS. 10A and 10B show the measurement results of the pulse waves from the right ankle shown in FIG. 10A and the left ankle shown in FIG. 10B of the healthy subject. FIGS. 11 and 12 show the step response for the right upper arm to the right ankle (the right step response) and the step response for the left upper arm to the left ankle (the left step response) calculated from the measurement results in FIGS. 10A and 10B and the pulse waves measured in the right and left upper arms, respectively. The comparison of these responses in FIG. 13 shows that they are nearly identical.

FIG. 14 is an X-ray image showing the arterial condition of the patient with arteriosclerosis obliterans. FIG. 14 shows an arterial occlusion in the circled region.

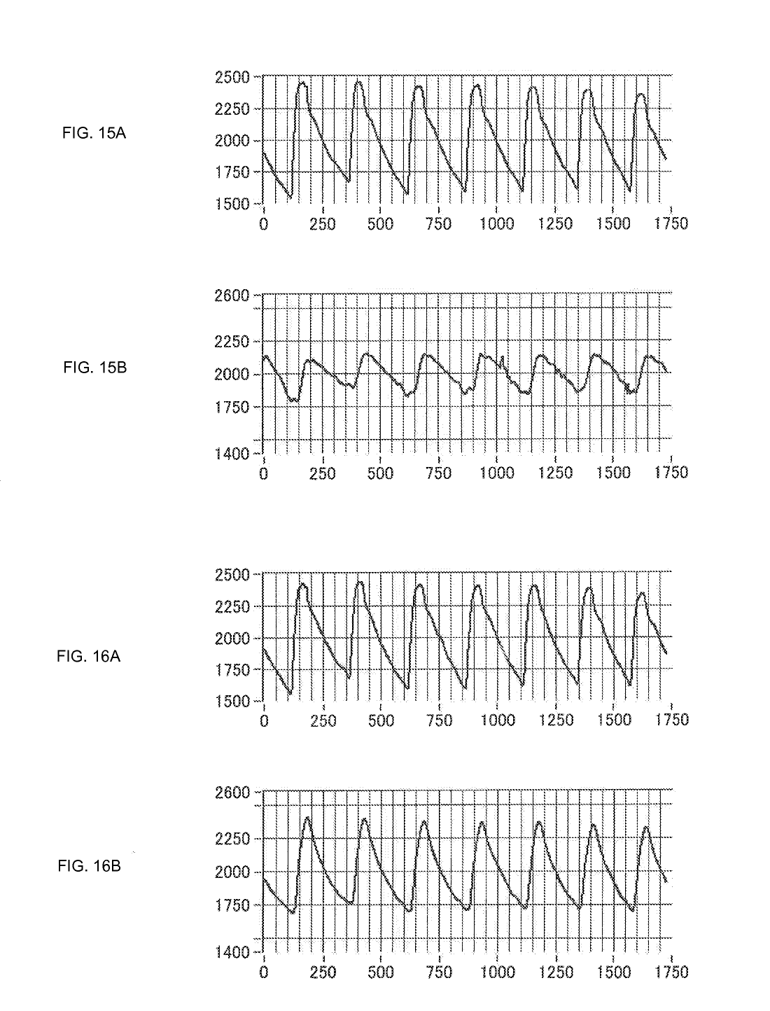

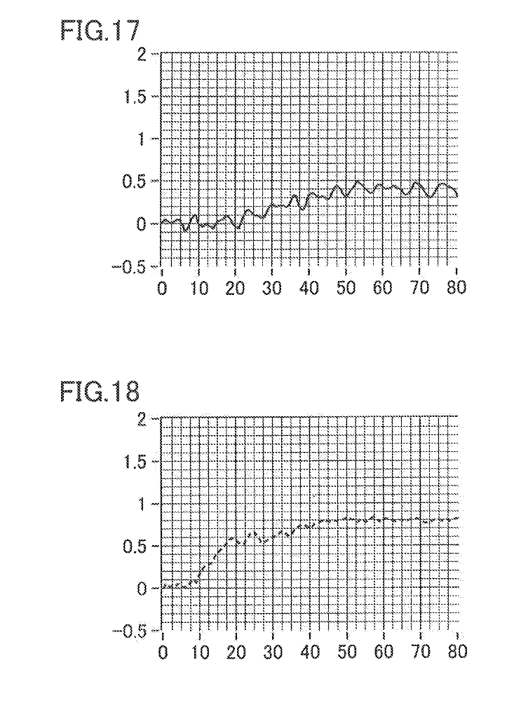

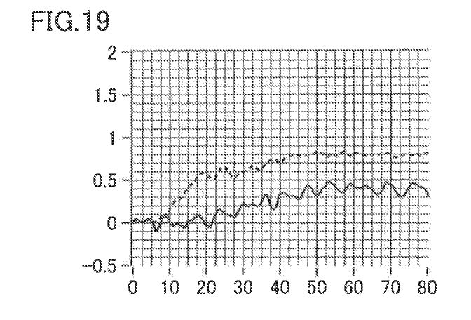

FIGS. 15A and 15B show the measurement results of the pulse waves in the right upper arm shown in FIG. 15A and the right ankle shown in FIG. 15B of the patient, and FIGS. 16A and 16B show the measurement results of the pulse waves in the left upper arm shown in FIG. 16A and the left ankle shown in FIG. 16B of the patient. FIGS. 17 and 18 show the right step response calculated from the pulse waves measured in the right upper arm and the right ankle in FIGS. 15A and 15B and the left step response calculated from the pulse waves measured in the left upper arm and the left ankle in FIGS. 16A and 16B, respectively. As clearly seen in FIG. 19, the comparison of these responses show that they are greatly different from each other.

Based on the above, it is safe to say that the higher the correlation between the right and left step responses is, the lower the possibility of an arterial occlusion is, and that the lower the correlation between the right and left step responses is, the higher the possibility of arteriosclerosis is.

In the light of the above, the inventors of the present invention calculated degrees of arteriostenosis and variations in step responses using a circulatory system model. The circulatory system model employed by the inventors represents the vascular system of a body divided into multiple segments. One exemplary circulatory system model is the so-called “Avolio Model” described in Reference Document 1, “Avolio, A.P, Multi-branched Model of Human Arterial System, 1980, Med. & Biol. Eng. & Comp., 18, 796.” The inventors used the Avolio Model as the circulatory system model for the calculations.

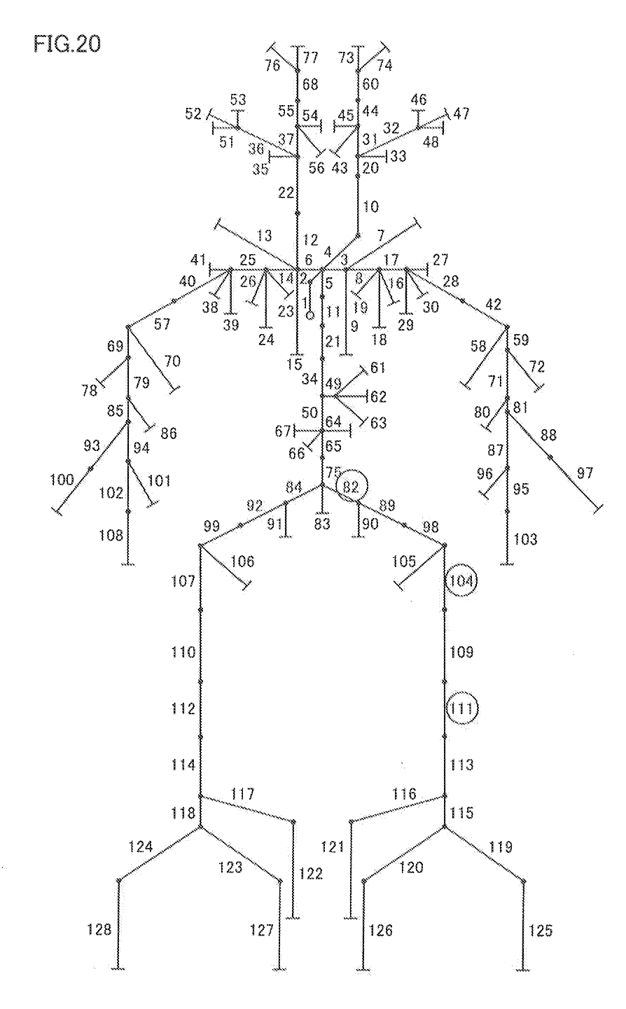

FIG. 20 is a schematic diagram of the Avolio Model. Referring to FIG. 20, the Avolio Model divides the systemic arteries into 128 vascular elements (segments) and defines geometric values that represent the respective segments. In the Avolio model, the geometric values include a length, a radius, a vessel wall thickness, and a Young's modulus associated with the respective segments.

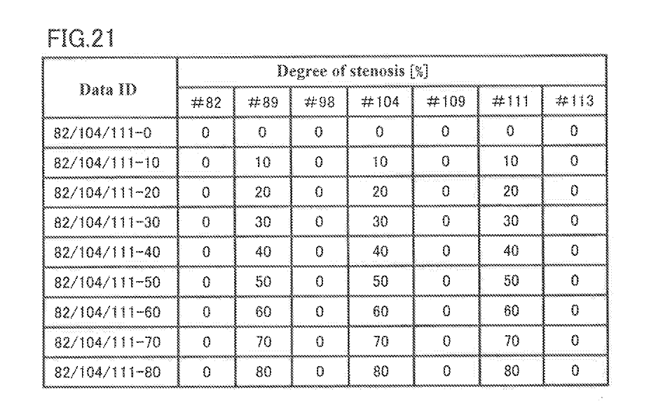

In the Avolio Model of FIG. 20, the inventors of the present application set parameters to create stenosis in the segments designated by the element numbers 82, 104, and 111 (circled in FIG. 20) in varying degrees and calculated the variations in the step responses. FIG. 21 is a table showing the degrees of stenosis created in the segments designated by the element numbers 82, 104, and 111 (circled in FIG. 20) in the Avolio Model that were used by the inventors to perform calculations. The degree of stenosis designated by Data ID “82/104/111-0” is set to zero percent for all of the segments so as to simulate or calculate the step response of the healthy subject. The larger the data ID number is, the greater the degree of stenosis in the segment is, thus producing a step response for the more advanced arteriosclerosis.

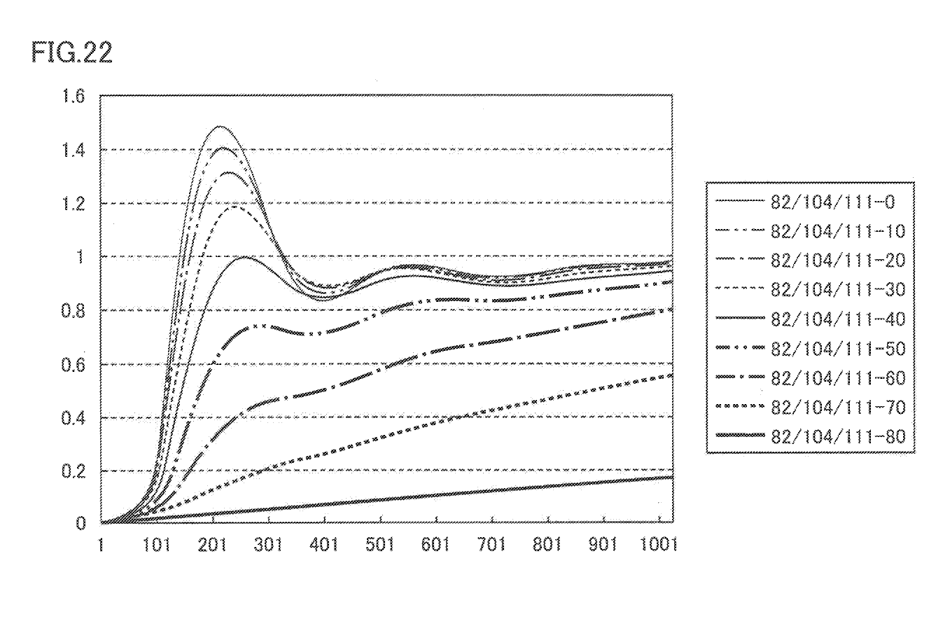

FIG. 22 is a graph plotting the results of the calculations. FIG. 22 shows that the healthier the subject is, the steeper the rising is and the more rapidly the response drops after reaching the maximum, and that the greater the degree of stenosis in the subject, the gentler the rising is and the smaller the change in the response becomes after reaching the maximum.

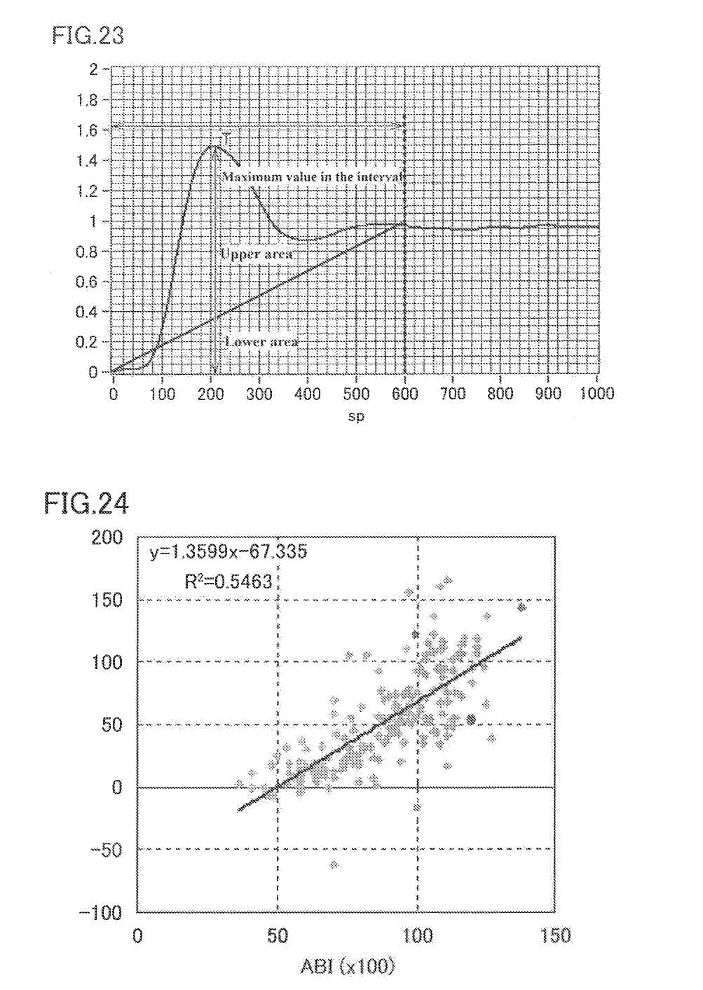

In the light of the foregoing, as shown in FIG. 23, the inventors of the present application defined an upper area, the ratio of the upper area to the lower area, and the maximum value in the step response interval and examined whether or not these three values may be used as the foregoing second index.

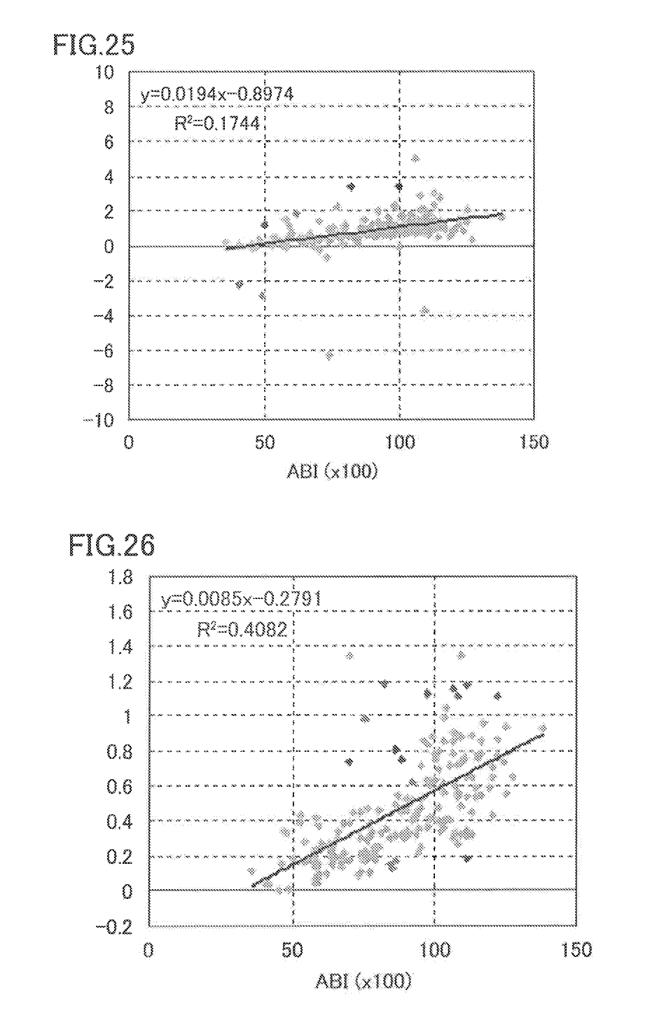

FIGS. 24-26 are graphs showing correlations between the ABI and the upper area, the ratio of the upper area to the lower area, and the maximum value in the interval, respectively. The measurements used for this purpose were the measurement results from the 200 adult males and females that were used to verify the correlations in FIGS. 3-5.

FIGS. 24-26 confirm that a certain degree of correlation exists between the ABI and any of the values and verify that a particularly strong correlation exists between the ABI and the upper area. It is therefore thought that the values obtained from the step response may be used as a second index and in particular, the upper area calculated from the step response may be used as the second index for evaluating the reliability of the ABI. Alternatively, it is also considered possible to use a combination of at least two of % MAP, UT, and pulse amplitude, and the index calculated from the step response as the second index in order to enhance the correlation.

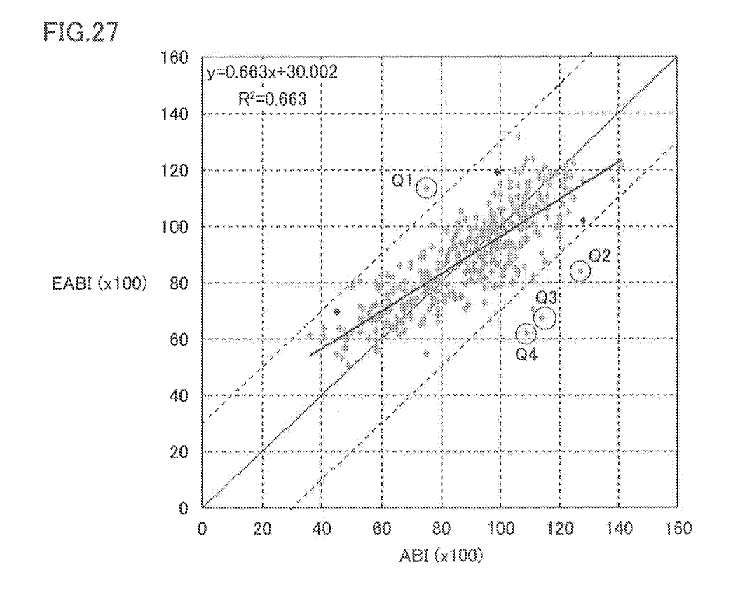

As an example, the inventors of the present application calculated, as the second index, a value (EABI) by multiplying each of % MAP (A), UT (B), pulse amplitude (C) and the index calculated from the step response (D) (e.g., the upper area) by a conversion factor, and examined the correlation between the second index and the ABI, i.e., the first index. In particular, the second index was obtained according to the formula, EABI=aA+bB+cC+dD+e (where a-e are coefficients), so as to compare this index with the ABI. FIG. 27 is a graph showing a correlation between the ABI and the EABI.

FIG. 27 verifies a relatively strong degree of correlation between the ABI and the second index calculated by combining % MAP (A), UT (B), pulse amplitude (C), and the index calculated from the step response (D) (e.g., the upper area), and FIG. 27 also verifies that this correlation is stronger than the correlation between the ABI and any one of or a combination of % MAP, UT, and pulse amplitude.

As in the cases discussed in relation to FIG. 6, there are several measurements that greatly deviate from the regression line as indicated by Q1-Q4 in FIG. 27. As in FIG. 6, examination of these measurement results shows the reliability of the blood pressure measurements is low for all these cases. For this reason, the correlation is likely to be even stronger if these cases are excluded.

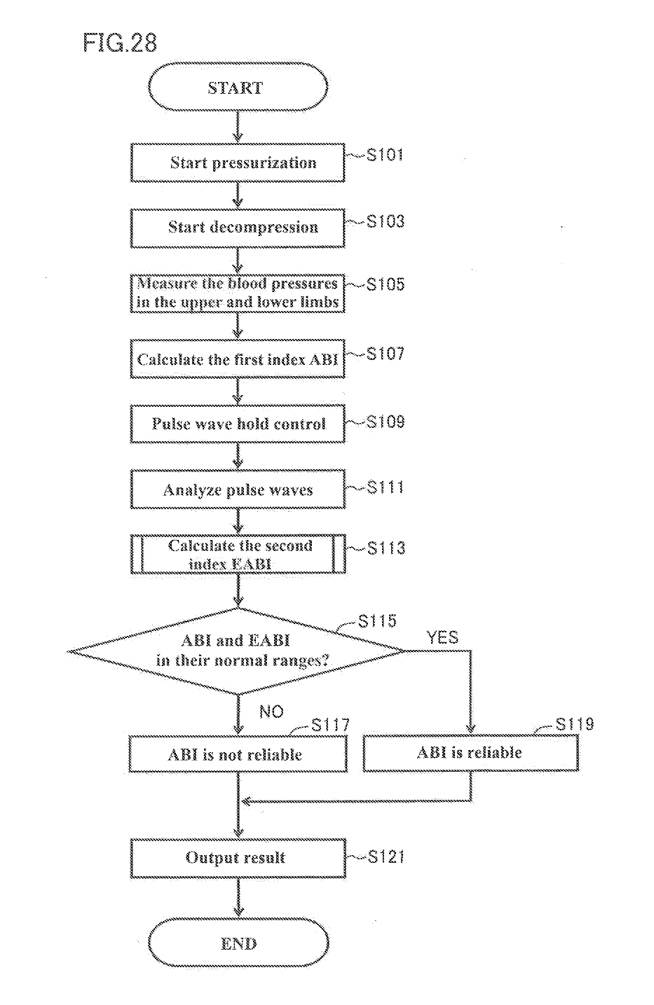

FIG. 28 is a flowchart representing a specific example of the operational flow that occurs in the measurement device 100. The operation represented in the flowchart of FIG. 28 is carried out on the CPU 10 as the CPU 10 reads out and executes a program stored in the ROM 12 while using the RAM 14 as the work memory so as to execute the functionalities shown in FIG. 2.

Referring to FIG. 28, the CPU 10 starts pressurization of the cuffs 24 in Step S101, and once a predetermined cuff pressure is reached, the CPU 10 starts decompression in Step S103. The predetermined cuff pressure, which is at least higher than a typical maximal blood pressure, may be a prescribed value or a value obtained by adding a predetermined pressure to an estimated maximal blood pressure during the process of pressurization.

Subsequently, in Step S105, the CPU 10 measures the blood pressures in the upper and lower limbs based on the variations of the cuff pressures during the pressurization of the cuffs 24. In Step S107, the CPU 10 calculates the ABI or the first index using these measurements.

Once the blood pressures are measured, the CPU 10 performs hold control to maintain the cuff pressures in the range suitable for measuring pulse waves in Step S109. Such suitable pressure values may be constant pressures of about 50-60 mmHg and pressures that are about 5-10 mmHg lower than the minimal blood pressure, for example. The CPU 10 analyzes the pulse waves obtained based on the variations in the cuff pressures under hold control in Step S111 and, in Step S113, calculates the index value used as the second index to evaluate the reliability of the ABI calculated as the first index.

The CPU 10 stores in advance a normal range of the ABI and a normal range of the EABI or the second index. The CPU 10 compares the ABI calculated in Step S107 and the EABI calculated in Step S113 with their respective normal ranges stored in advance. If the result of the comparison indicates that they are both in their respective normal ranges (YES in Step S115), the CPU 10 determines in Step S119 that the reliability of the ABI calculated in Step S107 is high.

Conversely, if even one of the ABI and the EABI is outside of its normal range, the CPU 10 determines in Step S117 that the reliability of the ABI calculated in Step S107 is low.

An alternative method for evaluating the reliability of the ABI may include comparing the ABI calculated in Step S107 with the EABI calculated in Step S113, and determine that the reliability of the ABI is high if the ABI and the EABI coincide with each other or fall in a predetermined range and determine that the reliability of the ABI is low if otherwise.

In Step S121, the CPU 10 outputs the result of determination as well as the ABI, which was calculated as the first index. This output may be displayed on a screen or transmitted to a separate device, such as a PC or an external recording medium. The outputted result of determination may include the ABI along with a message or symbol denoting the degree of reliability of the ABI. Alternatively, the manner of outputting the result (e.g., the manner of display) may be varied according to the result of determination or the ABI may be outputted along with the calculated second index value as the result of determination.

It should be noted that there are a variety of methods that can be employed to calculate the second index in the foregoing Step S113. This is because, as described above, any one or a combination of at least two of % MAP, UT, pulse amplitude, and a lower limb-upper limb pulse wave transfer function (e.g., the upper area) may be used as the second index value.

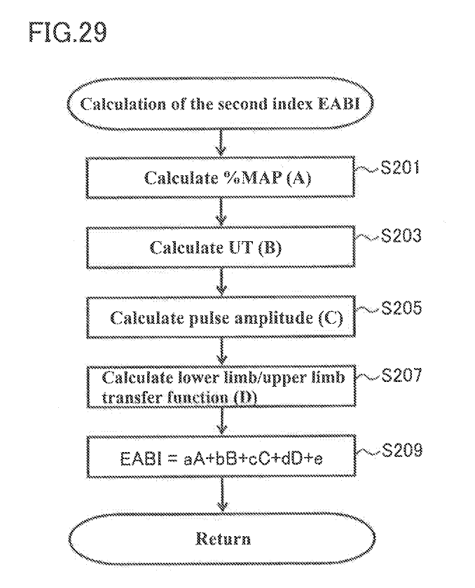

For example, FIG. 29 shows a flowchart representing a specific example of the operation in the foregoing Step S113 in which all of the above are combined for the calculation of the second index value. As described above, the second index calculated in this manner has a high correlation with the first index, thus resulting in highly accurate determination of the reliability.

With reference to FIG. 29, in Steps S201-207, the CPU 10 calculates % MAP (A), UT (B), pulse amplitude (C), and a lower limb-upper limb pulse wave transfer function (D) (e.g., the upper area) in order. Note that the order of calculations is not limited to that shown in FIG. 29.

Subsequently, in Step S209, the CPU 10 uses a prescribed conversion factor to calculate the second index, EABI=aA+bB+cC+dD+e (where a-e are coefficients).

By performing the foregoing operation, the measurement device 100 implements a simple method of evaluating the reliability of the ABI, which is calculated as an index that indicates the presence or absence of stenosis in the arteries or the degree of stenosis in a subject. Accordingly, the foregoing preferred embodiment provides doctors and other medical practitioners with a useful device to determine the presence or absence of stenosis in the arteries or the degree of stenosis in a subject using the ABI.

It is possible to accurately determine the reliability of the ABI by using any one of the index values obtained from pulse waves (% MAP, UT, pulse amplitude, and a lower limb-upper limb pulse wave transfer function (e.g., the upper area)). However, a combination of these provides for more accurate determination of the reliability of the ABI. Moreover, as a result of their investigation, the inventors of the present application have verified that the use of a lower limb-upper limb pulse wave transfer function (e.g., the upper area), alone or in combination, provides for particularly accurate determination of the reliability of the ABI.

Furthermore, a program that causes the measurement device 100 or an arithmetic unit such as a personal computer (upon obtaining values/data from the measurement device 100) to calculate the foregoing second index and/or use the second index to determine the reliability of the ABI may also be provided. Such a program may be provided as a program product by storing the program on a computer-readable recording medium, such as a flexible disk, a CD-ROM (compact disk-read only memory), a ROM (read only memory), a RAM (random access memory), and a memory card associated with a computer. Also, such a program can be recorded on a computer-readable recording medium included in a computer, such as a hard disk, and provided as a program product. Moreover, the program may be provided by allowing it to be downloaded via a network.

Note that the program according to a preferred embodiment of the present invention may invoke necessary modules, among program modules provided as part of a computer operating system (OS), in a predetermined sequence at predetermined timings, and cause such modules to perform processing. In this case, processing is executed in cooperation with the OS, without the above modules being included in the program itself. Such a program that does not include such modules can also be the program according to a preferred embodiment of the present invention.

Also, the program according to the present invention may be provided incorporated in part of another program. In this case as well, processing is executed in cooperation with the other program, with the modules of the other program not included in the program itself. Such a program incorporated in another program can also be the program according to a preferred embodiment of the present invention.

The program product that is provided is executed after being installed in a program storage unit such as a hard disk. Note that the program product includes the program itself and the recording medium on which the program is stored.

The preferred embodiments of the present invention described above are to be considered in all respects only to be illustrative and not restrictive. The scope of the invention is indicated by the appended claims rather than the above description, and all changes which come within the meaning and range of equivalency of the claims are to be encompassed within the scope of the invention.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1-10. (canceled)

11. A measurement device for measuring biological values and calculating an Ankle Brachial Blood Pressure Index as an index of arteriostenosis from the biological values, the measurement device comprising:
a first cuff configured to be worn on an upper limb of a subject;
a second cuff configured to be worn on a lower limb of the subject;
a first sensor that detects an internal pressure of the first cuff;
a second sensor that detects an internal pressure of the second cuff;
an adjustment unit that adjusts the internal pressures of the first and second cuffs;
an arithmetic unit connected to the first and second sensors that measures the biological values from detection values detected by the first and second sensors and that performs operations to calculate an index using the biological values; and
an output device connected to the arithmetic unit and that outputs results of the operations performed by the arithmetic unit; wherein
the arithmetic unit includes:
a blood pressure measurement device that measures a blood pressure in the upper limb using the detection values obtained by the first sensor and measures a blood pressure in the lower limb using the detection values obtained by the second sensor;
a pulse wave measurement device that measures pulse waves in the upper limb using the detection values obtained by the first sensor and that measures pulse waves in the lower limb using the detection values obtained by the second sensor;
a first calculation device that calculates the Ankle Brachial Blood Pressure Index by calculating a ratio of the blood pressures in the upper and lower limbs;
a second calculation device that calculates a determination index used for evaluation of the Ankle Brachial Blood Pressure Index using the pulse waves in the upper and lower limbs;
an evaluation device that evaluates the reliability of the Ankle Brachial Blood Pressure Index using the Ankle Brachial Blood Pressure Index calculated by the first calculation device and the determination index calculated by the second calculation device; and
an output device that outputs the Ankle Brachial Blood Pressure Index along with a result of evaluation performed by the evaluation device.

12. A measurement device according to claim 11, wherein the evaluation device evaluates the reliability of the Ankle Brachial Blood Pressure Index by determining whether or not each of the Ankle Brachial Blood Pressure Index and the determination index is in a prescribed range thereof.

13. A measurement device according to claim 12, wherein the evaluation device evaluates the reliability of the Ankle Brachial Blood Pressure Index as high if the Ankle Brachial Blood Pressure Index and the determination index are both in their respective prescribed ranges and evaluates the reliability of the Ankle Brachial Blood Pressure Index as low if otherwise.

14. A measurement device according to claim 11, wherein the evaluation device evaluates the reliability of the Ankle Brachial Blood Pressure Index as high if the Ankle Brachial Blood Pressure Index is in a prescribed range from the determination index and evaluates the reliability of the Ankle Brachial Blood Pressure Index as low if otherwise.

15. A measurement device according to claim 11, wherein the determination index uses at least one of: a normalized pulse wave area, which is an index representing sharpness of a pulse wave; an upstroke time, which is an index representing a rising feature value of an ankle pulse wave; a pulse amplitude; and an index value representing a lower limb-upper limb pulse wave transfer function, which is a function for transfer of a pulse wave from the upper limb to the lower limb.

16. A measurement device according to claim 15, wherein the determination index is calculated by combining at least two of the normalized pulse wave area, the upstroke time, the pulse amplitude, and the index value representing a lower limb-upper limb pulse wave transfer function.

17. A measurement device according to claim 15, wherein the determination index is calculated by combining an index value representing a lower limb-upper limb pulse wave transfer function with at least one of the normalized pulse wave area, the upstroke time, and the pulse amplitude.

18. A measurement device according to claim 11, wherein the output device outputs an estimate value of the Ankle Brachial Blood Pressure Index calculated by second calculation device along with the Ankle Brachial Blood Pressure Index.

19. An evaluation method for evaluating reliability of an Ankle Brachial Blood Pressure Index as an index of arteriostenosis, the Ankle Brachial Blood Pressure Index being calculated from biological values, the method comprising the steps of:
obtaining the Ankle Brachial Blood Pressure Index calculated as a ratio of blood pressures in an upper limb and a lower limb of a subject;
calculating a determination index used to evaluate the Ankle Brachial Blood Pressure Index using pulse waves in the upper and lower limbs;
evaluating the reliability of the Ankle Brachial Blood Pressure Index using the Ankle Brachial Blood Pressure Index and the determination index; and
outputting the Ankle Brachial Blood Pressure Index to an output device along with a result of evaluation.

20. A non-transitory computer readable medium including an evaluation program to cause a computer to perform operations to evaluate reliability of an Ankle Brachial Blood Pressure Index as an index of arteriostenosis, the Ankle Brachial Blood Pressure Index being calculated from biological values, the program causing the computer to perform the steps of:
obtaining the Ankle Brachial Blood Pressure Index calculated as a ratio of blood pressures in an upper limb and a lower limb of a subject;
calculating a determination index used to evaluate the Ankle Brachial Blood Pressure Index using pulse waves in the upper and lower limbs;
evaluating the reliability of the Ankle Brachial Blood Pressure Index using the Ankle Brachial Blood Pressure Index and the determination index; and
outputting the Ankle Brachial Blood Pressure Index to an output device along with a result of evaluation.
